SEQUENCE LISTING (5 sequences)

SEQ ID NO: 1 - DNA, 2048 bp, synthetic construct
  source 1..2048: mol_type = other DNA
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatga

SEQ ID NO: 2 - DNA, 20 bp, synthetic construct
  source 1..20: mol_type = other DNA
cgagatccctccaaaatcaa

SEQ ID NO: 3 - DNA, 20 bp, synthetic construct
  source 1..20: mol_type = other DNA
atccacagtcttctgggtgg

SEQ ID NO: 4 - DNA, 20 bp, synthetic construct
  source 1..20: mol_type = other DNA
tcttgctgtggactggattg

SEQ ID NO: 5 - DNA, 20 bp, synthetic construct
  source 1..20: mol_type = other DNA
agcaattcctcgtggttgag

(19) United States
(12) Patent Application Publication
(10) Pub. No.: US 2025/0121031 A1
(43) Pub. Date: Apr. 17, 2025

(54) EGF-RICH EXOSOMES, PREPARATION METHOD AND USE THEREOF

(71) Applicant: Centre for Regenerative Medicine and Health, Hong Kong Institute of Science & Innovation, Chinese Ac, Pak Shek Kok (HK)

(72) Inventors: Dajiang QIN, Pak Shek Kok (HK); Hongjie XU, Pak Shek Kok (HK); Di WU, Pak Shek Kok (HK); Yixia LING, Pak Shek Kok (HK); Xia WANG, Pak Shek Kok (HK)

(21) Appl. No.: 18/927,230

(22) Filed: Oct. 25, 2024

(30) Foreign Application Priority Data
Oct. 26, 2023 (CN) 202311402066.9

(51) Int. Cl. A61K 38/18; A61K 9/50; C12N 5/0775; C12N 15/86

(52) U.S. Cl. CPC A61K 38/1808; A61K 9/5068; C12N 5/0668; C12N 15/86; C12N 2740/15043

(57) ABSTRACT

The disclosure provides an exosome. The exosome contains Epidermal Growth Factors (EGF) with increased content. The disclosure also provides a preparation method for the exosome. The exosome can be used for the treatment of EGF-related diseases, such as repair and healing of wound surfaces caused by corneal injury, burns, scalds, ulcers, surgeries and the like, and can be used for the prevention of scar hyperplasia.

### CROSS-REFERENCE TO RELATED APPLICATIONS

This application claims the priority of CN patent application No. 2023114020669, filed Oct. 26, 2023, the content of which is incorporated herein by reference in its entirety.

### SEQUENCE LISTING

The instant application contains a Sequence Listing which has been submitted in an XML file with the USPTO and is hereby incorporated by reference in its entirety. The Sequence Listing was created on Oct. 10, 2024, is named “SequenceListing.xml”, and is 7,316 bytes in size.

### TECHNICAL FIELD

The disclosure belongs to the field of biotechnology and specifically relates to an Epidermal Growth Factor (EGF)-rich exosome as well as a preparation method and use thereof.

### BACKGROUND ART

Epidermal growth factor (EGF) is a type I transmembrane glycoprotein with a molecular weight of 134 kD. The coding gene of the human EGF protein is located on the human chromosome 4 and is a polypeptide consisting of 1207 amino acids, which is a typical member of the growth factor family. It is widely expressed in various tissues and organs such as the kidney, pancreas, salivary gland, skin, thyroid gland, heart, gallbladder, etc., and plays an extremely important role in the pathophysiological process of human body. EGF is a pleiotropic protein with multiple binding sites, which can play various biological roles, including having various biological functions, particularly having significant effects on the proliferation, differentiation, and migration of cells, thus playing an important role in the development of organogenesis and wound repair.

In recent years, EGF has received increasing attention mainly in the field of ulcer and skin wound healing. When EGF binds to the receptor, the downstream signaling pathway is activated, and it produces a variety of biological effects, including: strong mitogenic activity on a variety of cells from different sources, such as epidermal cells, epithelial cells, and fibroblasts, promoting synthesis of intracellular DNA, RNA, and protein, capable of accelerating proliferation and differentiation of tissue; promoting the proliferation and differentiation of embryonic cells and influencing organogenesis and development; and promoting the proliferation of mesenchymal cells, such as corneal endothelial cells, which has a certain value in the repair and treatment of cornea-related diseases. However, although EGF has the effects of treating skin repair in many directions, EGF itself is a multifunctional protein, has too sizeable molecular weight to be easily absorbed, and presents significant challenges in pharmaceutical development, so it is difficult to use in clinical treatment. Therefore, it is necessary to develop a targeted therapeutic medicine.

Exosomes are nanoscale extracellular vesicles (30 to 150 nm) that are exocytosed by cells and belong to one of the paracrine mediators of cells. Exosomes may include RNA, proteins, and other molecules, which are packaged by the parental cells and sent to the connected cells for intercellular communication. Due to the advantages of low immunogenicity, low toxicity, and the ability to cross biological barriers such as the blood-brain barrier, it is possible for exosomes to deliver these contents to the receptor cells, which can help the proteins to perform their functions and regulate the physiological functions. Currently, medicines based on exosomes enter clinical drug candidates to treat various diseases. However, the content of EGF carried in exosomes secreted by mesenchymal stem cells in the prior art is very low, it is necessary to develop an exosome that can increase the content of EGF.

### SUMMARY OF THE INVENTION

The disclosure provides an Epidermal Growth Factor (EGF)-rich exosome and the preparation method thereof. By genetically editing mesenchymal stem cells to improve their basal ability to synthesize EGF and secrete it and identifying and confirming the encapsulation of a high content of EGF in the exosomes through a variety of methods, the disclosure solves the problem that exosomes contain only a low content of EGF.

In one aspect, the disclosure provides an exosome comprising an increased content of EGF.

In some embodiments, the EGF content of the exosome increases by more than 20 times, for example, more than 21 times, more than 22 times, more than 23 times, more than 24 times, more than 25 times, more than 26 times, more than 27 times, more than 28 times, more than 29 times, more than 30 times, more than 31 times, more than 32 times, more than 33 times, more than 34 times, or more than 35 times. In some specific embodiments, the EGF content of the exosomes increases by more than 35 times.

In some embodiments, the increased EGF content of the exosome is compared with that of the exosomes of wild-type mesenchymal stem cells.

In some embodiments, the exosome may be produced by host cells, such as mammalian cells. In some embodiments, the host cell may be generated from stem cells. In some embodiments, the host cell may include, but is not limited to, a human embryonic kidney (HEK) cell, a Chinese hamster ovary (CHO) cell, an HT-1080 cell, a HeLa cell, a PERC-6 cell, a CEVEC cell, a fibroblast, an amniotic cell, an epithelial cell, and a mesenchymal stem cell (MSC). In a preferred embodiment, the host cell may be MSC. In a specific embodiment, the MSC may include a bone marrow-derived MSC, an adipose-derived MSC, and an umbilical cord blood-derived MSC.

The disclosure also provides a method for the preparation of the exosome. The method comprises the following steps: (1) introducing a nucleotide sequence encoding EGF into a host cell; and (2) isolating the exosome from a culture medium of the host cell.

In some embodiments, the nucleic acid sequence encoding EGF is located in a lentiviral vector. In a specific embodiment, the lentiviral vector comprises a 5′LTR containing ψ sequence, a 3′LTR, a target gene sequence between 5′LTR and 3′LTR, and a promoter sequence and a translation initiation sequence operably linked to the target gene sequence.

In some embodiments, the nucleotide sequence encoding EGF is a nucleotide sequence shown in SEQ ID NO: 1 or a nucleotide sequence having at least 85%, at least 88%, at least 90%, at least 92%, at least 95%, at least 97%, at least 98%, at least 99%, or 100% sequence identity to SEQ ID NO: 1.

In some embodiments, in step (1), the lentiviral vector is used to infect the host cell.

In some embodiments, the 3′LTR and 5′LTR comprise one or more modifications.

In some embodiments, the U3 of the 5′LTR and 3′LTR may be deleted or mutated.

In some embodiments, the 3′LTR is a 3′LTR in which a U3 region is deleted (3′LTR-ΔU3).

In some embodiments, the 5′LTR is a 5′LTR in which a U3 region is deleted (5′LTR-ΔU3).

In some embodiments, the promoter of the 5′LTR is selected from the group consisting of a cytomegalovirus CMV promoter, a Rous sarcoma virus RSV promoter, and a simian virus SV40 promoter, preferably the Rous sarcoma virus RSV promoter.

In some embodiments, the promoter operably linked to the nucleotide encoding EGF is selected from the group consisting of a short elongation factor 1A (EF1α) promoter or a transcriptionally active fragment thereof, an RSV promoter, and a simian virus SV40 promoter, preferably a short elongation factor 1A (EF1α) promoter.

In some embodiments, the nucleotide encoding EGF is operably linked to an EF1a promoter and a Kozak translation initiation sequence.

In some embodiments, the lentiviral vector further comprises a nucleotide encoding a screening marker.

In some embodiments, the screening marker is selected from one or more of a Luciferase, a fluorescent protein, a streptavidin binding peptide, a puromycin resistance marker, an ampicillin resistance marker, a kanamycin resistance marker, and a neomycin resistance marker. Preferably, the screening marker is an enhanced green fluorescent protein (EGFP).

In some embodiments, the lentiviral vector further comprises an SV40 early pA.

In some embodiments, a post-transcriptional regulatory element of the lentiviral vector comprises a woodchuck hepatitis virus post-transcriptional regulatory element (WPRE).

In some embodiments, the lentiviral vector comprises a retroviral export element. “Retroviral export element” refers to a cis-acting post-transcriptional regulatory element that regulates the transport of RNA transcripts from the nucleus to the cytoplasm. Preferably, the retroviral export element includes, but is not limited to, a human immunodeficiency virus (HIV) rev response element (RRE) and a hepatitis B virus post-transcriptional regulatory element (HPRE).

In some embodiments, the lentiviral vector further comprises a central polypurine region (cPPT) or a central termination sequence (CTS).

Preferably, the cPPT sequence is a cPPT of HIV1.

Preferably, the CTS sequence is a CTS of HIV1.

In some embodiments, the lentiviral vector comprises sequentially from the 5′LTR region to the 3′LTR region: an RSV promoter, a 5′LTR-ΔU3, a ψ sequence, an RRE, a cPPT, an EF1a promoter, a Kozak translation initiation sequence, a nucleotide encoding EGF, an EGFP, a WRPE, a 3′LTR-ΔU3, and an SV40 early pA.

In some embodiments, the host cell can express a target nucleotide carried in the lentiviral vector.

In some embodiments, the host cell is a mammalian cell. In some embodiments, the host cell may include, but is not limited to, a human embryonic kidney (HEK) cell, a Chinese hamster ovary (CHO) cell, an HT-1080 cell, a HeLa cell, a PERC-6 cell, a CEVEC cell, a fibroblast, an amniotic cell, an epithelial cell, and a mesenchymal stem cell (MSC).

In some embodiments, the host cell may be a stem cell. In some preferred embodiments, the host cell may be a mesenchymal stem cell. In some specific embodiments, the MSC may comprise bone marrow-derived MSC, adipose-derived MSC, and umbilical cord blood-derived MSC.

In some embodiments, the stem cell is a human umbilical cord mesenchymal stem cell transfected with the lentiviral vector carrying the nucleotide encoding EGF.

In some embodiments, in step (2), the seeding density of the stem cell is 1.0×10⁵ to 2.0×10⁵, preferably 1.0×10⁵.

In some embodiments, in step (2), the multiplicity of infection (MOI) of a virus is 10 to 30, preferably 20 to 30.

In some embodiments, in step (2), the auxiliary transfection reagent used for transfection is polybrene. Preferably, a working concentration of Polybrene is 5 to 8 μg/mL.

In some embodiments, the method further comprises extracting exosomes by an ultracentrifugation process, filtration centrifugation, density gradient centrifugation process, immunomagnetic bead method, or PS affinity method. Compared with other exosome extraction methods, the ultracentrifugation process has an advantage of obtaining more exosomes, and therefore, the ultracentrifugation process is a more commonly used exosome extraction method.

In some embodiments, the exosome prepared by the method has a high expression of EGF. Thus, the EGF-rich exosome can be obtained by the method of the disclosure.

The disclosure also provides a pharmaceutical composition, comprising the aforementioned exosome or an exosome obtained by the aforementioned preparation method, and a pharmaceutically acceptable excipient or carrier.

The disclosure also provides use of the aforementioned exosome, the exosome prepared by the aforementioned method, or the aforementioned pharmaceutical composition in the preparation of a medicament for the treatment of wound surface repair and healing, tissue repair, or wound healing, or for the prevention of scar hyperplasia.

In some embodiments, the wound surface includes, but is not limited to, a wound surface caused by corneal injury, burn, scald, surgery, or chronic ulcer, and the like.

In some embodiments, the exosome prepared by the disclosure can be used to accelerate wound healing, improve the quality of wound recovery, reduce scar area, and the like.

The disclosure has at least one of the following beneficial effects:

1. The lentiviral vector overexpressing EGF involved in the disclosure is designed to be simple and efficient with high infection efficiency.

2. The exosome of a mesenchymal stem cell infected with the lentiviral vector overexpressing EGF involved in the disclosure is enriched in an increased EGF.

3. The exosome prepared in the disclosure belongs to a vesicle-encapsulated protein, which is more conducive to anti-inflammatory factor proteins reaching an inflammatory cell and thus being absorbed by the inflammatory cell than a pure protein; Compared with the cell therapy with MSCs, the exosome of the disclosure has the advantages such as non-immunogenicity and good permeability.

### BRIEF DESCRIPTION OF THE DRAWINGS

FIG. 1 shows a mapping of an overexpressing lentiviral vector.

FIG. 2 shows the transfection effect of hUC-MSCs transfected with the lentivirus observed by confocal microscopy.

FIG. 3 shows the results of GFP expression detected by flow cytometry.

FIG. 4 shows the morphologies of the exosome of MSC with overexpressing empty vector as well as the exosome of MSC with overexpressing EGF identified by electron microscopy.

FIG. 5 shows the contents of EGF in the exosome of MSC with overexpressing empty vector as well as the exosome of MSC with overexpressing EGF detected by ELISA, ***P≤0.001, compared with the MSC-Exo group.

### DETAILED EMBODIMENTS OF THE INVENTION

In order to elucidate the purpose, technical solution, and advantages of the disclosure, a further detailed explanation will be provided hereinafter in connection with examples. The specific examples described herein are only for illustrative purposes of the disclosure and are not intended to limit the scope of the disclosure. Furthermore, in the following explanation, the description of the well-known structures and techniques is omitted to avoid unnecessarily confusing the concepts of the disclosure. Such structures and techniques are also described in numerous publications.

### Definitions

Unless otherwise defined, all technical terms and terms of art used in this invention have the same meaning as commonly used in the field to which this invention belongs. For the purpose of illustrating this description, the following definitions will be applied, and where appropriate, the term used in a singular form will also include the plural form and vice versa.

Unless the context clearly indicates otherwise, the expressions “a” and “an” as used herein include plural references. For example, the reference to “a cell” includes multiple such cells and equivalents known to those skilled in the art.

Exosomes are membranous vesicles released into the extracellular matrix by the fusion of an intracellular multivesicular body (MVB) with the cell membrane. Almost all types of cells can produce and release exosomes. It is a nanoscale lipid encapsulation structure with a diameter of 30 to 150 nm, which contains substances such as proteins, mRNA, and microRNA inside. Exosomes are naturally present in body fluids, including blood, saliva, urine, and breast milk, and the secreted exosomes may enter body fluids such as blood, saliva, urine, and breast milk and reach other cells and tissues through the circulatory system, producing remote regulatory effects. Most exosomes contain MHC class I and/or II molecules, heat shock proteins (hsp), tetraspanins, integrins, cytoskeletal proteins, mRNA, and other non-coding RNA, as well as various metabolic enzymes. The membrane of exosomes contains different surface markers, typically the tetraspanins CD9, CD63, and CD81. The tetraspanins can be used in the diagnosis of various tumors and infectious diseases.

A lentiviral vector system comprises a packaging plasmid, an envelope plasmid, and a vector plasmid, and can be either a two-plasmid system, a three-plasmid system, or a four-plasmid system. The three-plasmid system is a system in which the cis-acting sequence structures required for packaging, reverse transcription, and integration are separated from sequences encoding trans-acting proteins in the lentiviral genome and cloned into three separate plasmids, with all of the auxiliary sequences being removed. The four-plasmid system is obtained by modifying the three-plasmid system. Compared with the three-plasmid system, the first change is that the rev gene is placed on a separate expression plasmid, and the addition of a new plasmid increases the safety of the system. The second change is that the tat gene is removed, and a chimeric 5′LTR fused to a heterologous promoter is added to the vector plasmid to initiate expression of the vector plasmid. Additionally, the three-plasmid system and the four-plasmid system also have a vector in which the target gene sequence can be placed, called a vector plasmid.

Epidermal growth factor (EGF) is a prototypical member of the EGF superfamily of peptide growth factors. The precoding protein is subjected to a proteolytic treatment to generate a 53-amino acid epidermal growth factor peptide, which constitutes a single-chain polypeptide of 53 amino acid residues. EGF is a precursor consisting of 1,217 amino acid residues, and a mature EGF is generated from its precursor by proteolysis. EGF is a potent mitogenic factor that acts through high-affinity binding to the cell surface receptor, epidermal growth factor receptor (EGFR/ErbB).

A ψ sequence is the minimal packaging signal required for the capsidation of lentiviral genomes.

“Stem cell” refers to an undifferentiated cell that is capable of long-term self-renewal or of producing at least one identical copy of the initial cell; differentiating into multiple cells at the single-cell level, and in some cases, only a single specific cell type; and achieving functional regeneration of tissues in vivo. Stem cells are subdivided into totipotent, sub-totipotent, pluripotent, and oligopotent stem cells based on their developmental potential.

Mesenchymal stem cells (MSCs) have been widely used in clinical research of regenerative medicine and autoimmune diseases due to their multidirectional differentiation potential and immunomodulatory functions. Unlike other stem cells, such as hematopoietic stem cells, MSCs are a class of stem cells capable of amplification in vitro. In this application, UMSCs refer to MSCs obtained from umbilical cords, particularly human umbilical cord mesenchymal stem cells (hUC-MSCs). Human umbilical cord mesenchymal stem cells are mesenchymal stem cells originating from the umbilical cord of a newborn baby and have strong proliferative and multidirectional differentiation capacity. However, it should be understood by those skilled in the art that the source of MSCs is not limited to human umbilical cord MSCs, and that other sources of MSCs can be used in the disclosure.

The following embodiments and drawings are provided below to assist in understanding the disclosure. It should be understood, however, that these embodiments and the drawings are intended to illustrate the disclosure only, but do not constitute any limitation. The actual scope of the disclosure is elaborated in the claims. It should be understood that any modifications and changes may be made without departing from the spirit of the disclosure.

### Example 1: Preparation of Recombinant Stem Cell

### 1. Plasmid Construction

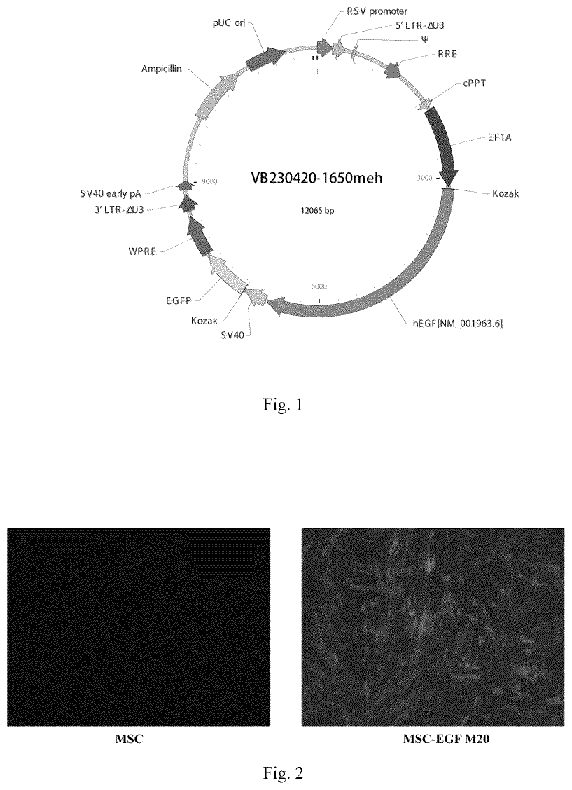

Firstly, an EGF-overexpressing lentiviral vector was designed and synthesized, and the vector design mapping is shown in FIG. 1. The lentiviral vector contains sequentially from the 5′LTR region to the 3′LTR region: RSV promoter, 5′LTR-ΔU3, ψ-sequence, RRE, cPPT, EF1α promoter, Kozak translation initiation sequence, nucleotides encoding EGF, EGFP, WRPE, 3′LTR-ΔU3 and SV40 early pA. The vector combined the EF1α promoter sequence and the Kozak translation initiation sequence in upstream of the inserted EGF gene (SEQ ID NO: 1), which ensured the high expression of the viral plasmid in stem cells. The constructed lentiviral vectors also carried an enhanced GFP sequence to facilitate the observation of transfection efficiency. Due to the long EGF sequence, the above elements have reached the maximum carrying capacity of the lentivirus, and it is neither possible to carry the sequences of the drug screening marker antibiotic Puro, which are routinely required to be retained, nor to add additional sequences of the signal peptide, linker region or transmembrane region.

The insertion sequence and restriction endonuclease sites on the vector backbone were analyzed, and the enzyme that could cut the appropriate bands was selected to digest the plasmid DNA. The digested DNA was subjected to agarose gel electrophoresis, and was stained with ethidium bromide EtBr, and the sizes of the DNA fragments were determined, and primers targeting the vector backbone and/or the insertion sequence were designed. The nucleic acid sequence was identified by Sanger sequencing and the alignment was entirely correct. The viral vector plasmid was successfully constructed.

### 2. Infection Methods

After the successful construction of the viral vector plasmid, viral packaging was performed, and then hUC-MSCs were transfected. On DO of transduction, hUC-MSCs of the P4 to P8 generation were selected, and the cells were seeded into 6-well plates at a density of 1×10⁵ to 2×10⁵/well, with the culture medium of DMEM/F12+10% FBS, and cultured in an incubator at 37° C. 5% CO₂ for 18 to 20 h. The transfection was performed after the cell density reached 30% to 50% of confluence. On the day of transduction (D1), the viral solution was thawed on ice and mixed gently. The count of viruses was aspirated according to the MOI (MOI was about 20) and added to the culture medium and mixed gently. It was suitable that the amount of culture medium covered the surface area of the culture medium and the amount was 100 μL/mL, and the amount of culture medium used in a 6-well plate was 1 mL/well. The original culture medium was aspirated out and the culture medium added with viruses was added to the 6-well plate on which hUC-MSCs were cultured. At the same time, 5 to 8 μg/mL of an auxiliary transfection reagent, polybrene, was added to each well, and mixed well so that the virus covered every cell, and the recombinant stem cells MSC-EGF were prepared by culturing in the incubator at 37° C. 5% CO₂ for 6 to 8 h. Excessive exposure to polybrene can cause cell toxicity, so the transduction time should not be too long, otherwise the cell status may be affected. Meanwhile, the virus solution with empty vector was used as a blank control for transduction in the same way, and wild-type MSCs of the blank control group were prepared.

### Example 2: Identification of Infection Efficiency

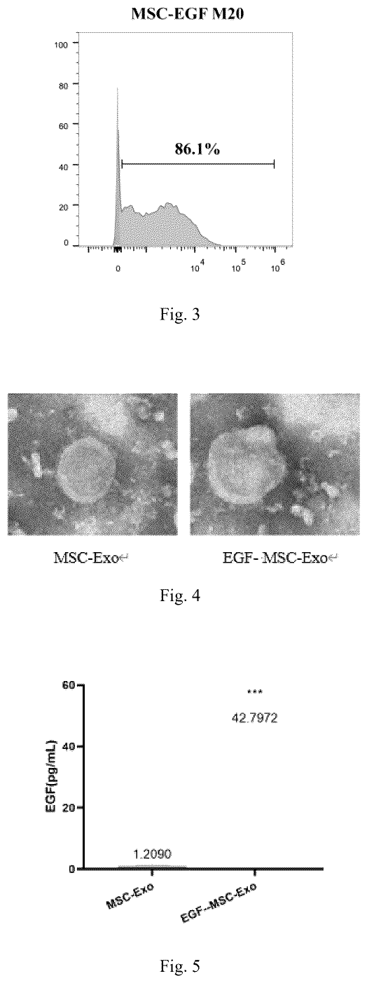

On the second day after transduction, the culture medium containing viruses was aspirated, and fresh DMEM/F12+10% FBS culture medium was added and cultured overnight in the incubator at 37° C. 5% CO₂. Usually, the genes carried by lentivirus began to be expressed on the second day of transduction, and green fluorescence can be observed 48 to 72 h after transfection. Fluorescent expression was observed daily, and strong expression of green fluorescent protein of GFP was observed by confocal microscopy after 72 h (see FIG. 2). The percentage of green fluorescence was counted, while the efficiency of infection was confirmed by flow cytometry. The results showed that the effective infection rate exceeded 86% when MOI was 20 (see FIG. 3).

### Example 3: Preparation and Characterization of Exosomes

Wild-type mesenchymal cells (MSC) from blank control group and umbilical cord mesenchymal stem cells overexpressing EGF (EGF-MSC) transfected with virus prepared in Example 1 were amplified in 15 cm dishes using a serum-containing culture medium. The serum-containing culture medium was replaced with a serum-free substrate culture medium 40 hours before the collection of culture medium. After culturing the two types of overexpressing MSCs in serum-free culture medium for 40 hours, the culture medium was collected, and the cells were lysed with RIPA lysis solution supplemented with protease inhibitors.

The supernatant was collected by centrifugating at 2000 g to remove cells and dead cells. The supernatant was subjected to a second centrifugation at 10,000 g of centrifugal forces to remove cellular debris and the resulting supernatant was collected. The resulting supernatant was then subjected to a third centrifugation at 167,000 g of centrifugal forces to obtain a precipitate, namely the exosome. Finally, the precipitate was washed with PBS and subjected to a fourth centrifugation at 167,000 of centrifugal forces. The resulting supernatant was removed and the resulting precipitate was resuspended by adding into 200 μL of PBS to give an exosome of umbilical cord MSC overexpressing cell line.

The extracted exosome was detected by Western Blot (WB), and it was found that both the exosome extracted from MSCs overexpressing EGF (MSC-Exo) and the exosome extracted from MSCs overexpressing EGF (EGF-MSC-Exo) contained common markers of exosomes, such as CD63, CD81, CD9, and TSG101. By preparing electron microscopy samples and performing an electron microscopy analysis, the exosomes extracted from MSCs overexpressing empty vector and MSCs overexpressing EGF were found to present a vesicle-like pattern, which was in conformity with the morphology of exosomes (see FIG. 4).

### Example 4: Identification of Exosome Secretion Levels

To confirm the increased expression of EGF in the paracrine exosome from MSCs, an enzyme-linked immunosorbent assay (ELISA) was used for analysis.

The exosome from wild-type mesenchymal cells (MSCs) from blank control group and the exosome from the mesenchymal stem cells (EGF-MSC) infected by the lentivirus integrated with EGF sequence prepared in Example 1 were resuspended with appropriate PBS to obtain a sample. 100 μL of the sample and different concentrations of standards were added to a plate coated with antibodies, with three wells per group. A blank control group was set up. The plate was sealed with a sealing tape and incubated at 37° C. for 90 min and then was washed with 1× washing buffer for 5 times. The plate was added with a biotinylated antibody working solution at 50 μL/well, covered with sealing film, and incubated at 37° C. for 90 min. The liquid in the well was removed, and then the plate was washed with 1× washing buffer for 5 times. The plate was added with horseradish peroxidase-conjugated streptavidin-HRP working solution at 100 μL/well, covered with sealing film, and incubated at 37° C. for 30 min. The plate was washed for 5 times. The plate was added with TMB chromogenic solution at 100 μL/well, and incubated at 37° C. for 15 min. The reaction was terminated by adding termination solution at 100 μL/well. The concentration was calculated by reading the values at a detection wavelength of 450 nm.

As shown in FIG. 5, the results indicated that the exosome extracted from the mesenchymal stem cells (MSC-EGF) infected with EGF lentivirus contained a significantly increased content of EGF compared with that of wild-type mesenchymal stem cells (MSCs), in which EGF protein secretion was increased from 1.2 pg/mL prior to infection to over 42.8 pg/mL, and the secretion level was increased by more than 35 times.

The technical solutions of the disclosure are not limited to the above specific examples, and any technical variants made based on the technical solutions of the disclosure fall within the scope of the disclosure.

What is claimed is:

1. An exosome, comprising an increased content of epidermal growth factor (EGF).

2. The exosome of claim 1, wherein the EGF content of the exosome is increased by more than 20 times.

3. A method for the preparation of the exosome of claim 1, comprising the following steps: (1) introducing a nucleotide sequence encoding EGF into a host cell; and (2) isolating the exosome from a culture medium of the host cell.

4. The method of claim 3, wherein the nucleic acid sequence encoding EGF is located in a lentiviral vector, wherein the lentiviral vector comprises a 5′ LTR containing ψ sequence, a 3′ LTR, a target gene sequence between 5′ LTR and 3′ LTR, and a promoter sequence and a translation initiation sequence operably linked to the target gene sequence.

5. The method of claim 3, wherein the nucleotide sequence encoding EGF is a nucleotide sequence shown in SEQ ID NO: 1 or a nucleotide sequence having at least 85%, at least 90% or at least 95% sequence identity to SEQ ID NO: 1.

6. The method of claim 3, wherein the promoter of the 5′LTR is selected from the group consisting of a cytomegalovirus CMV promoter, a Rous sarcoma virus RSV promoter, and a simian virus SV40 promoter; and/or
the promoter operably linked to the nucleotide sequence encoding EGF is selected from the group consisting of a short elongation factor 1A (EF1α) promoter or a transcriptionally active fragment thereof, an RSV promoter, and a simian virus SV40 promoter.

7. The method of claim 6, wherein the promoter operably linked to the nucleotide sequence encoding EGF is a short elongation factor 1A (EF1α) promoter.

8. The method of claim 6, wherein the promoter of the 5′LTR is a Rous sarcoma virus RSV promoter.

9. The method of claim 6, wherein the nucleotide sequence encoding EGF is operably linked to an EF1α promoter and a Kozak translation initiation sequence.

10. The method of claim 3, wherein the lentiviral vector further comprises a nucleotide encoding a screening marker, a woodchuck hepatitis virus post-transcriptional regulatory element WPRE, a retroviral export element, and a central polypurine region (cPPT) or a central termination sequence (CTS).

11. The method of claim 10, wherein the screening marker is selected from one or more of a Luciferase, a fluorescent protein, a streptavidin binding peptide, a puromycin resistance marker, an ampicillin resistance marker, a kanamycin resistance marker, and a neomycin resistance marker.

12. The method of claim 10, wherein the screening marker is an enhanced green fluorescent protein (EGFP).

13. The method of claim 10, wherein the retroviral export element is selected from the group consisting of a human immunodeficiency virus rev response element RRE and a hepatitis B virus post-transcriptional regulatory element HPRE.

14. The method of claim 3, wherein the lentiviral vector comprises sequentially from the 5′LTR region to the 3′LTR region: an RSV promoter, a 5′LTR-ΔU3, a ψ-sequence, an RRE, a cPPT, an EF1α promoter, a Kozak translation initiation sequence, a nucleotide encoding EGF, a EGFP, a WRPE, a 3′LTR-ΔU3, and an SV40 early pA.

15. The method of claim 3, wherein the host cell is a stem cell.

16. The method of claim 15, wherein the stem cell is a mesenchymal stem cell.

17. The method of claim 16, wherein the mesenchymal stem cell is a human umbilical cord mesenchymal stem cell.

18. A pharmaceutical composition, wherein the pharmaceutical composition comprises an exosome of claim 1 and a pharmaceutically acceptable excipient or carrier.

19. A method for the treatment of wound surface repair and healing, tissue repair, or wound healing, or for the prevention of scar hyperplasia in a subject in need thereof, comprising administrating to the subject a therapeutically effective amount of the exosome of claim 1 or a pharmaceutical composition that comprises the exosome.

20. The method of claim 19, wherein the wound surfaces include wound surfaces caused by corneal injury, burns, scalds, surgeries, or chronic ulcers.
